(19) United States
(12) Patent Application Publication
(10) Pub. No.: US 2024/0164406 A1
(43) Pub. Date: May 23, 2024

(54) METHODS OF PRODUCING MEAT ANALOGUE FOOD INGREDIENTS

(71) Applicant: Solar Foods Oy, Lappeenranta (FI)

(72) Inventors: Anna HÄKÄMIES, Lappeenranta (FI); Anni NISOV, Espoo (FI)

(73) Assignee: Solar Foods Oy, Lappeenranta (FI)

(21) Appl. No.: 18/551,904

(22) PCT Filed: Apr. 7, 2022
(86) PCT No.: PCT/FI2022/050225

§ 371 Date: Sep. 22, 2023

(30) Foreign Application Priority Data
Apr. 27, 2021 (FI) 20215489

(51) Int. Cl. A23J 3/22; A23J 1/00; A23J 3/14; A23J 3/16; A23J 3/18; A23J 3/20; A23J 3/26; C12N 1/20

(52) U.S. Cl. CPC A23J 3/227; A23J 1/008; A23J 3/14; A23J 3/16; A23J 3/18; A23J 3/20; A23J 3/26; C12N 1/20

(57) ABSTRACT

A method of producing a meat analogue food ingredient. The method includes a downstream process including cultivating bacterial cells to obtain a biomass, separating a liquid phase and a solid phase of the biomass, concentrating the biomass by removing the liquid phase; and drying the biomass to obtain the first protein powder; mixing a first protein powder with a liquid and NaCl to obtain a powder mixture; extruding the powder mixture with high-moisture extrusion; cutting the extruded mixture; and cooling the extruded mixture.

### TECHNICAL FIELD

The present disclosure relates generally to meat-analogues; and more specifically to methods of producing meat analogue food ingredients.

### BACKGROUND

A balanced human diet requires proteins, carbohydrates, fats, vitamins and minerals in proper proportions. In human diet, plants (such as for example soy) and animals (such as for example cattle, pig, poultry, fish) have been the potential sources of the above-identified nutrients, especially high-quality proteins. Currently, an ever-increasing demand for high-quality proteins while a limited availability of land for growing plants and rearing of animals possess challenge in meeting the protein-requirements of the exponentially growing population of the world.

Moreover, animal-based proteins do not appeal to a wide demographic of consumers identified as vegetarians or vegans, and some non-vegetarians seeking to reduce their meat consumption. Therefore, it was required for the global food industry to adapt to comparatively more sustainable and healthier alternatives for animal-based meat products.

Typical alternatives for animal-based product include meat analogues (namely, artificial meat) produced from plants such as soybeans, corn, peanut, and the like. In this regard, the plants may be grown naturally or by using three-dimensional (3D) printing techniques for generating plant-based meat analogues. However, the plant-based meat analogues fail to satisfactorily mimic the standard meat in terms of appearance, texture, flavour, chewiness and juiciness thereof. For example, with plant-based meat analogues it is difficult to achieve a fibrillar structure resembling meat fibers. Moreover, the plant-based meat analogues have a typical bean-off flavour that makes it difficult to flavour to imitate the meat-like flavour. Furthermore, the production of plant-based meat analogue is highly labour-intensive. Also, the plant-based meat analogues are poor in other nutrients, such as for example iron, vitamins, and so forth.

Recent advances in food technology has extended production of meat analogues using microbes such as yeast, algae and the like. In this regard, techniques such as cell culture followed by extrusion process, 3D-printing techniques, and so forth have been employed to produce microbe-based meat analogues. However, a specific food-grade 3D-printing equipment is not always available and is rather expensive. Moreover, the microbe-based meat analogues, like the plant-based meat analogues, lack meat-like texture and other characteristics, are not suitable for consumption by mammals, such as humans and animals, mostly due to the poor digestibility thereof. Furthermore, poor digestibility may be associated with low nutrient availability from such microbe-based meat analogues. Also, microbe-based meat analogues may be the biggest source of endotoxin content in human (or livestock) daily diet. Normally, when such a meat analogue is ingested, the epithelial cells of bowels act as a physical barrier with the production of a mucus layer that prevents the endotoxins from translocating into the bloodstream. However, in case of endotoxemia or leaky gut syndrome, endotoxins translocate to bloodstreams due to mucosal degradation, and result in health risks ranging from allergies to fatal toxic reactions.

Therefore, in light of the foregoing discussion, there exists a need to overcome drawbacks associated with conventional techniques of producing the meat analogue food ingredient that has meat-like texture and improved digestibility.

### SUMMARY

The present disclosure seeks to provide a method of producing a meat analogue food ingredient. The present disclosure seeks to provide a solution to the existing problem of producing meat analogue food ingredient from microbes. An aim of the present disclosure is to provide a solution that overcomes at least partially the problems encountered in prior art.

In an aspect, an embodiment of the present disclosure provides a method of producing a meat analogue food ingredient, the method comprising:
a downstream process comprising
   cultivating bacterial cells to obtain a biomass,
   separating a liquid phase and a solid phase of the biomass,
   concentrating the biomass by removing the liquid phase, and
   drying the biomass to obtain the first protein powder;
mixing a first protein powder with a liquid and NaCl to obtain a powder mixture;
extruding the powder mixture with high-moisture extrusion;
cutting the extruded mixture; and
cooling the extruded mixture.

Embodiments of the present disclosure substantially eliminate or at least partially address the aforementioned problems in the prior art, and provides an efficient and robust method of producing the meat analogue food ingredient that imitates meat-like texture and is digestible by mammals, such as for example human and animals.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1 is a flowchart depicting steps of a method of producing a meat analogue food ingredient, in accordance with an embodiment of the present disclosure; and

FIGS. 2, 3 and 4 are flowcharts illustrating upstream and downstream processing of meat analogue food ingredient, in accordance with various embodiments of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

In one aspect, an embodiment of the present disclosure provides a method of producing a meat analogue food ingredient, the method comprising:
a downstream process comprising
   cultivating bacterial cells to obtain a biomass,
   separating a liquid phase and a solid phase of the biomass,
   concentrating the biomass by removing the liquid phase, and
   drying the biomass to obtain the first protein powder;
mixing a first protein powder with a liquid and NaCl to obtain a powder mixture;
extruding the powder mixture with high-moisture extrusion;
cutting the extruded mixture; and
cooling the extruded mixture.

The present disclosure provides the aforementioned method of producing the meat analogue food ingredient. The method of the present disclosure comprises utilizing protein powder derived from microbial biomass, mixed with a liquid, salt and spices, and producing meat analogue food ingredient using extrusion process. Beneficially, the method is efficient and less labour-intensive. Moreover, the method provides a healthier alternative to the standard meat, in terms of appearance, texture, flavour and nutrition column. Furthermore, the meat analogue food ingredient produced using the aforesaid method is animal-free, and therefore, is suitable for vegetarian and vegan consumers. Additionally, beneficially, the meat analogue food ingredient is readily digestible by humans and animals and supplies them with high-quality protein, iron and vitamins such as B12.

Throughout the present disclosure, the term “meat analogue food ingredient” as used herein refers to a meat-like product made from animal-free products. Typically, the meat analogue food ingredient is derived from plants or microbes, for example. Generally, the meat analogue food ingredient could be used as a complete food or an ingredient in food, typically, due to certain aesthetic qualities (such as texture, appearance, flavour, for example) or chemical characteristics (such as a protein content, for example) that resemble specific types of animal-based meat. It will be appreciated that the meat analogue food ingredient is a more sustainable, healthier and cruelty-free alternative to standard animal-based meat obtained after sacrificing animals. Moreover, meat analogue food alternative appeals to a wide demographic of consumers identified as vegetarians or vegans, and some non-vegetarians seeking to reduce their meat consumption. Furthermore, the production of meat analogue food ingredient contributes negligibly to the global warming effect as compared to the production of animal-based meat that releases large amounts of carbon dioxide in the environment.

Throughout the present disclosure, the term “protein powder” as used herein refers to a nutritional supplement, extracted from plants and/or microbes for example, in dehydrated form. Generally, the protein powder provides a concentrated source of proteins with no or negligible carbohydrates, fats or any other compounds. Alternatively, the protein powder comprises proteins and could be fortified with compounds such as vitamins and minerals, such as calcium, iron, and so forth. It will be appreciated that proteins are the essential for muscle building and recovery. Therefore, protein consumption should be monitored to supply the required amount of proteins in the diet, while avoiding long-term excessive protein intake that affects kidneys, liver and body's bone-and-calcium balance. Optionally, protein powder can be mixed with water, milk, fruit or vegetable juices or smoothies, and the like for consumption by a human or an animal (including birds, fishes, and the like). More optionally, the protein powder could be used to produce meat analogue food ingredient, as discussed hereforth.

The method comprises a downstream process, which initiates with cultivating the bacterial cells (namely, the inoculum) to obtain a biomass. The term “biomass” as used herein refers to a measure of amount of living component (namely, bacteria) in a sample. Notably, the biomass comprises a solid phase (i.e. bacterial cells) and a liquid phase (growth medium). The bacterial cells may be cultivated (namely, cultured) by gas fermentation or by sugar fermentation in a media suspension (comprising a carbon source, a nitrogen source, an energy source, minerals and other specific nutrients) within vessels called bioreactors under controlled conditions (such as temperature, humidity, pH, and any of an aerobic, anaerobic or facultative condition, for example). Optionally, the bacterial cells are cultivated (namely, cultured) by gas fermentation and the feed comprises at least one of selected from CO₂, CH₄, H₂, O₂, NH₃, at least one mineral. Optionally, the biomass could be produced in continuous or batch cultivation of the bacterial cells. It will be appreciated that microbes have shorter reproduction time and, thus, can be grown rapidly to produce high cell density biomass. Beneficially, the high cell density of the biomass is sufficient for production of protein powder for consumption by humans for example. Additionally, beneficially, large-scale production of biomass and a harvesting thereof is easier and cost efficient as compared to harvesting protein from a single bacterial cell due to the need for highly efficient micro-scale laboratory equipment.

Moreover, the cultivated biomass having a high cell density is harvested and further subjected to processing steps, such as incubation, separation, homogenization and drying for example, to obtain the desired final product.

The method may also comprise an upstream process before downstream process. Typically, the upstream process comprises creating an optimum environment for the microbial cells, such as for example the bacterial cells, to grow and make the desired intracellular protein(s). Optionally, the upstream process comprises genetically engineering the microbial cells to produce high yield of the desired protein and/or other nutritional components, such as antioxidants, iron, vitamins, and so forth. It will be appreciated that one or more batches of bacterial cells that make the desired intracellular protein(s) are selected as a starting material or an inoculum for further growth thereof. The term “downstream processing” as used herein refers to the process that follows the selection of bacterial cells making high yield of protein. Typically, the downstream processing are unit operations that facilitate production of the final product in a manner useful for the consumers (humans or animals) thereof. In this regard, the downstream processing comprises subjecting the bacterial cells to physiological, chemical and mechanical conditions, to provide a final product that is suitable and safe for use by the consumers.

The downstream processing comprises separating the liquid phase and the solid phase of the biomass and concentrating the biomass by removing the liquid phase. Optionally, separating is carried out with a separation method selected from at least one of a centrifugation, a filtration. Centrifugation is typically a technique for the separation of particles according to their size, shape, density, viscosity or speed of rotor employed for separation. In this regard, the solution is placed in a centrifuge tube that is then placed in rotor and spun at a definite speed. Optionally, centrifugation is performed with a centrifugal force ranging between 10000×g and 20000×g. The centrifugation separates about 90-95% of liquid phase from the solid phase. It will be appreciated that centrifugation is the most efficient and easiest way to separate the liquid and solid phases. The filtration technique typically separates the liquid and solid phases through a semi-permeable membrane that allows the liquid phase to pass therethrough while retaining the solid phase over the said semi-permeable membrane. The filtration provides the most energy-efficient way to separate the liquid phase from the solid phase. It will be appreciated that along with the liquid phase, hydrolysed components of the cell wall structures including the endotoxins are removed from the concentrated biomass, thus, leaving the concentrated biomass with reduced endotoxins therein.

The downstream processing comprises drying the biomass to obtain a first protein powder. The term “drying” as used herein refers to a process of drying out liquids from raw materials, such as the biomass. Optionally, drying of biomass is accomplished by subjecting the biomass to either relatively low temperatures by rotating over the biomass in a closed system, such as a drying drum for example, or rapidly drying using a hot gas. The drying is typically carried out at temperatures ranging from 120, 125, 130 or 135° C. up to 125, 130, 135 or 140° C., and pressure ranging from 2, 2.5, 3 or 3.5 bars up to 2.5, 3, 3.5 or 4 bars. It will be appreciated that drying the biomass increases the dry matter content of the biomass, such as for example in a range from 96, 96.5, 97 or 97.5% up to 96.5, 97, 97.5 or 98%. Optionally, the drying is selected as at least one of a drum drying or a spray drying. Optionally, the dryer is selected to be at least one of a drum dryer or a spray dryer. Optionally, the drying process is followed by milling of the final product to obtain a powder form of the final product, i.e. the protein powder. Beneficially, drying at the aforesaid temperature range dries out liquid (or water) in the biomass to obtain powder form thereof that is easy to store. Moreover, drying the biomass increases the shelf-life of the biomass by preventing a potential infestation thereof by pathogens. Furthermore, drying the biomass facilitates efficient grinding thereof to obtain the final product with a desired particle size.

The method comprises mixing a first protein powder with a liquid and NaCl to obtain a powder mixture. The term “first protein powder” as used herein refers to dehydrated (or powdered) form of proteins derived from microbes, thus, commonly referred to as single cell proteins (or SCP). Dry matter content of the first protein powder is from 96% up to 98%. For example, the dry matter content of the protein powder is in a range from 96, 96.5, 97 or 97.5% up to 96.5, 97, 97.5 or 98%. It will be appreciated that the first protein powder typically comprises edible microbial cells. Beneficially, the first protein powder is a rich source of proteins as well as iron and vitamin, such as B12 for example. Moreover, the liquid and NaCl (or common salt) is mixed with the first protein powder to form a flavoured dough therefrom. Optionally, other salts, such as KCl, monosodium glutamate (MSG), and the like. Optionally, besides liquid and NaCl, spices and preservatives could be mixed with the first protein powder to mimic the meat-like flavour. It will be appreciated that the first protein powder, liquid, NaCl and other additives are all used under Good Manufacturing Practices.

Optionally, the downstream process further comprises incubating the biomass with a heat treatment at temperature from 55 ºC up to 75° C. from 15 minutes up to 40 minutes. Notably, incubating the biomass with heat treatment facilitates certain chemical and structural changes in the bacterial cells. Specifically, incubating facilitates disrupt the cell wall to release endotoxins, that could be harmful to the humans if they translocate from the gut into the bloodstream. Optionally, incubating is performed before the separating step. The incubation may for example be carried out at temperatures from 55, 56, 57, 58, 59, 60, 65 or 70ºC up to 56, 57, 58, 59, 60, 65, 70 or 75ºC for the incubation period from 15, 20, 25, 30 or 35 minutes up to 20, 25, 30, 35 or 40 minutes. Optionally, the heat-exchanger is selected to be at least one of a tank heat-exchanger, a tubular heat-exchanger, or a plate heat-exchanger. Beneficially, cell wall degradation as a result of incubation of biomass results in a final product with at least 10-1000 times lower endotoxin response. Additionally, incubation at the aforesaid temperature range prevents growth of unwanted microbes and result in a pure culture of only the desired bacteria.

Optionally, the downstream process further comprises homogenizing the bacterial cells of the biomass before drying step. Notably, homogenizing at least partially degrades cell walls of the bacterial cells. The term “homogenizing” as used herein refers to a means of physical disruption of the bacterial cell walls. It will be appreciated that incubating the bacterial cells partially disrupts their cell walls, and homogenizing the biomass further disrupts the cell walls. Typically, homogenizing exploits fluid flow, particle-particle interaction, and pressure drop to facilitate cell disruption. Beneficially, homogenizing results in partial lysis of bacterial cells and increasing soluble protein content of the biomass thereby improving functional properties of the biomass as a food ingredient. Typically, used homogenizing devices include mortar and pestle, blenders, bead mills, sonicators, rotor-stator, and the like. Additionally, homogenizing the biomass further removes endotoxines remaining in the concentrated biomass, thereby further reducing from the homogenized biomass.

Optionally, homogenizing could be carried out using a high-pressure homogenization (HPH) or a milling technique. The term “high-pressure homogenization” as used herein refers to a physical or mechanical process of forcing a stream of sample, such as the concentrated biomass, through a high-pressure homogenizing device to homogenize the sample and/or reduce the particle size of any components within the sample. Typically, the high-pressure homogenizing device subjects the sample to a plurality of forces, such as high pressure or any combination of shear forces for example. Optionally, the homogenizing is carried out at pressure from 800 bars up to 2000 bars for at least one run. The homogenization pressure may, for example, be from 800, 1000, 1200, 1400, 1600 or 1800 bars up to 1000, 1200, 1400, 1600, 1800 or 2000 bars. The term “at least one run” as used herein refers to the number of cycles or passes (such as once, twice or thrice) the concentrated biomass is subjected to increase cell disruption efficiency. Preferably, the homogenizing is carried out from 700 bars up to 1000 bars. The homogenization pressure may, for example, be from 700, 750, 800, 850, 900 or 950 bars up to 750, 800, 850, 900, 950 or 1000 bars. Furthermore, more preferably, the homogenizing is carried out at 900 bars. Beneficially, the said range of homogenization pressure provides best results with increased soluble protein content and decreased endotoxin levels in the homogenized biomass.

Optionally, the downstream process further comprises filtering the homogenized bacterial cells of the biomass by at least one of selected from nanofiltration or ultrafiltration. After homogenization, a biomass slurry is obtained. The biomass slurry is filtered with ultrafiltration to eliminate cell debris and with nanofiltration to concentrate the protein content in the biomass slurry. Alternatively, precipitating the homogenized biomass slurry can be used to increase the protein content in the biomass slurry. The filtering step can be carried out after breaking down the bacterial cells of the biomass by homogenizising. If cells are un-broken, there is nothing to filtrate from the biomass. Filtering by nanofiltration or ultrafiltration enables to increase protein content in the biomass. Beneficially, higher protein content of the biomass improves meat analogue food ingredient texture in extruding step and makes the meat analogues food ingredient more fibrous and more meat-like texture.

The term “milling” as used herein refers to mechanical ways to break the larger particle size components into smaller sizes, such as nano-sized particles. The milling is carried out by milling agents that exert shear forces to break the larger sized particles into smaller sized particles. Optionally, milling technique includes liquid milling (namely, bead milling or ball milling) and sonication. The bead mill homogenization techniques utilize beads inside a mill homogenizing device which are rapidly agitated to grind and homogenize the sample. It will be appreciated that cell wall disruption resulting from homogenizing the biomass removes the remaining endotoxines from the bacterial cells. Beneficially, the soluble protein content in the biomass is increased due to milling homogenization. Additionally, milling also results in decreased endotoxin levels in the biomass.

Optionally, the method further comprises adjusting the biomass pH to be from 7.4 up to 8.5 after at least one step of selected from separating the liquid phase or homogenizing. The pH may for example be from 7.4, 7.5, 7.6, 7.7, 7.8 or 7.9 up to 7.5, 7.6, 7.7, 7.8, 7.9, 8.0, 8.1, 8.2, 8.3, 8.4 or 8.5. It will be appreciated that a suitable pH is an essential factor for the growth media to facilitate bacterial growth. In this regard, acids and bases could be added to the concentrated biomass to adjust the pH thereof. Optionally, a pH adjustor could be selected from potassium hydroxide (KOH) or calcium hydroxide (Ca(OH)₂). It will be further appreciated that for pH lower than 7.0, the extruded product would fail to provide a meat-like texture upon extrusion.

Optionally, the liquid is selected to be at least one of a water, a protein slurry. The first protein powder mixed with water results in a dough, for example. Optionally, the water is a double-distilled water. The term “protein slurry” as used herein refers to a fluid comprising a solid phase, comprising essentially of proteins, and a liquid phase. Optionally, in addition to proteins, the solid phase of the protein slurry comprises carbohydrates, fats, dietary fibers, ash, and so forth. Optionally, the protein slurry comprises from 90% up to 95% of water, from 1% up to 10% of a third protein powder. The amount of water in the protein slurry may for example be from 90, 91, 92, 93 or 94% up to 91, 92, 93, 94 or 95% of the total amount of protein slurry, and the amount of the third protein powder may for example be from 1, 2, 3, 4, 5, 6, 7, 8 or 9% up to 2, 3, 4, 5, 6, 7, 8, 9 or 10%. In an example, the solid phase of the protein slurry is 6% and liquid is 94%. In an example, the protein slurry comprises 5% solid phase and 95% of water, wherein the solid phase comprises 65% proteins, 10% fat, 25% minerals and fibers. It is appreciated, That the best results are obtained with aforementioned ranges. Furthermore, the protein slurry with higher amount of water is too watery to obtain a meat like texture of the final product. Also, if the protein powder is present in higher amounts, the protein slurry is too thick for processing.

Optionally, the third protein powder comprises other proteins in powder form. The third protein powder could be different from the first protein powder in terms of structural and/or functional traits thereof. For example, the third protein powder could be an enzyme required for proper functioning of the first protein powder.

Optionally, the at least one of selected from the first protein powder and the third protein powder comprises an isolated bacterial strain deposited as VTT-E-193585 or a derivative thereof. The said isolated bacterial strain or a derivative thereof is typically a Gram-negative bacterium (which do not retain crystal violet stain used in the gram-staining method). It will be appreciated that the said isolated bacterial strain or a derivative thereof is genetically stable and can be grown in a broad range of process conditions, ranging from optimal to stressful conditions, over time. The term “genetically stable” as used herein, refers to a characteristic of a species or a strain/isolate to resist changes and maintain its genotype over multiple generations or cell divisions, ideally hundreds to thousands. Optionally, the said isolated bacterial strain or a derivative thereof utilize hydrogen gas as energy source and carbon dioxide as carbon source. Beneficially, the said strain or the derivative thereof comprises iron and vitamin B12. Moreover, the final product resulting from the said strain or the derivative thereof does not have a bean-off-flavor and is therefore easier to flavor. Possibly, the final product also has umami (namely, savory or “meat-like”) flavor.

Optionally, the powder mixture is mixed in a mixer selected from at least one of a pre-conditioner, a flour mixer, a twin-screw extrusion machine. It will be appreciated that the first protein powder, liquid and NaCl are mixed to obtain a homogenous mixture thereof. Moreover, mixing of the aforementioned constituents should ensure high retention of water by the first protein powder to enable softer and consistent final product. The pre-conditioner are mixers that heat, hydrate and homogenously blends the dry ingredients to yield a pre-treated product for further processing thereof, such as an extrusion thereof, for example. The flour mixer is enables large quantities of dough to be mixed. The flour mixer could be a standard kitchen equipment used for kneading dough using wheat flour, for example. The twin-screw extrusion machine is typically a system having a defined (or fixed) cross-section that is used to pass a material therethrough to provide a shape or the desired cross-section to the final product being extruded from the extrusion machine. In this regard, the extrusion machine uses friction (between the passing material and the extruder) and heat to due to pressure generated as a result of friction to shape the final product. Typically, the twin screw extrusion machine consists of two co-rotating screws arranged on shafts in a closed stationary barrel. Specifically, the twin-screw extrusion machine suitable for mixing, while extruding, highly viscous and rigid mixtures.

Moreover, the method comprises extruding the powder mixture with high-moisture extrusion. The term “extruding” as used herein refers to a process of shaping a material, such as a food product, into a product of a fixed cross-section (desirable form), such as slices, blocks, pieces, cubes, and so forth. In this regard, the material is forced through a die (namely, a perforated plate designed to produce the required shape), coupled to the given extruder, of the desired cross-section and subjected to compressive and shear stresses. The term “high-moisture extrusion” as used herein refers to a thermo-mechanical cooking process often used for production of high moisture products, such as high moisture meat analogues (HMMA). Typically, the high moisture extrusion process facilitates continuous mixing, kneading and shaping of the material being extruded. In this regard, the high moisture extrusion is carried out using a high moisture extrusion machine that employs heating of the barrel and shearing of the screws (such as the standard twin screw extrusion machine, as discussed above) to produce the HMMA. Alternatively, the powder mixture could be extruded using dry extrusion.

The HMMA typically comprise about 40%-70% moisture content, and thus, imitate meat-like texture and mouthfeel. Moreover, the HMMA may show fibrillar structure resembling meat fibers, for example. Beneficially, the HMMA offers a much improved fibrous and textured meat analogues as compared to conventional texturized vegetable proteins (TVP) that are produced using a low moisture extrusion process. Moreover, the HMMA could be mixed with other ingredients, such as spices, nutrients, pharmaceuticals and the like, to enhance the nutritional column and flavours of the HMMA. Furthermore, HMMA produced from dried biomass using high-moisture extrusion showed dramatically lowered endotoxin levels compared to the level thereof in the powder itself. High-moisture extrusion typically reduces the endotoxins from >4000 EU/g to <0.5 EU/g in the HMMA. Moreover, high-moisture extrusion of the protein powder that was produced without going through the downstream processing operations resulted in a HMMA which showed no endotoxic response.

Optionally, high-moisture extrusion is carried out with following parameters:
torque from 1.0 Nm up to 1.3 Nm;
die pressure from 15 bars up to 18 bars;
die temperature from 140° C. up to 160° C.; and
melting temperature from 135° C. up to 155 ºC.

In this regard, the term “torque” as used herein is typically the rotational force (namely, twisting force) between the shafts (onto which the screws are loaded) for causing the intermeshing shafts to co-rotate during the extrusion process. Optionally, the torque may for example be from 1.0, 1.1 or 1.2 Nm (abbreviated for Newton-meters) up to 1.1, 1.2 or 1.3 Nm. The term “die pressure” as used herein is typically the pressure generated at the extrusion machine front end that is coupled to the die. The die pressure may for example be from 15, 16 or 17 bars up to 16, 17 or 18 bars. The term “die temperature” as used herein is typically the temperature at the first end of the die as a result of the die pressure thereat. The die temperature may for example be from 140, 145, 150 or 155 up to 145, 150, 155° C. or 160° C. The term “melting temperature” as used herein is typically the temperature at which the product starts to melt. The melting temperature typically increases with the die pressure. The melting temperature may for example be from 135, 140, 145 or 150° C. up to 140, 145, 150 or 155° C. In an example, high moisture extrusion is carried out for a time duration of at least 5 minutes, at a torque of 1.2 Nm, die pressure of 14 bars, die temperature of 160 ºC, and melting temperature of 151 ºC. Notably, high die temperature and high melting temperature result in fibrous HMMA. It will be appreciated that the afore-mentioned conditions could be controlled based on the desired product to ensure uniformity of the final product.

Moreover, beneficially, the high moisture extrusion takes as input powder mixture and extrudes out the final product (namely HMMA) that has meat-like texture, additional nutrients as a result of addition of supplementary proteins and or nutrients, and/or higher digestibility as a result of addition of soluble fibers thereto. It will be appreciated that the steps of the method of producing meat analogues as disclosed so far could be altered (by way of addition or skipping) in order to produce final product of different quality. For example, the incubation and homogenization steps could be skipped in order to produce HMMA showing higher digestibility by the humans. Moreover, in the said example, addition of soluble fibers further increases the desirability of the said meat analogue.

It will be appreciated that extrusion breaks down microbial cell wall that improves digestibility. Optionally, the Digestible Indispensable Amino Acid Score (DIAAS) is shown to improve when the powder mixture was extruded. The DIAAS change is improves in a range from 0.22 to 0.79, as shown in Table 1 below. As shown, the non-incubated, non-homogenized powder mixture is reduced digestibility as accounted by the DIAAS of 0.22 and protein digestibility of 39, while the extrudate shows improved digestibility as accounted by the DIAAS of 0.79 and protein digestibility of 69. It will be appreciated that according to FAO recommendation, the protein is low quality if the DIAAS is lower than 0.75.

TABLE 1
Protein Digestibility score

| | | | | | Peak | | |
|---|---|---|---|---|---|---|---|
| | | | Calculated | | time for | | |
| | Total | Total | conversion | Protein | bioaccessible | | |
| | Nitrogen | AA | factor | digestibility | fraction | | Limiting |
| Sample | (g/100 g) | (g/100 g) | (cCF) | (%) | (min) | DIAAS | AA |
| Non | 11.55 | 57.4 | 4.97 | 39 | 30-60 | 0.22 | Trp |
| incubated; | | | | | | | |
| non- | | | | | | | |
| homogenized | | | | | | | |
| Extrudate | 4.98 | 22.6 | 4.54 | 67 | 60-90 | 0.79 | Trp |

Optionally, the method further comprises adding at least one of selected from at least one second protein powder and at least one soluble fiber to the powder mixture before mixing. The term “second protein powder” as used herein refers to dehydrated (or powdered) form of proteins derived from plants, for example. It will be appreciated that the second protein powder typically comprises edible plant isolates. The second protein powder generally aim to provide a holistic protein column that is not obtained from only the microbial proteins. Moreover, addition of the second protein powder provides better binding ability of the powder mixture and, therefore, ensures uniformity of the final product.

Optionally, the at least one second protein powder is selected to be at least one of a pea isolate powder, wheat gluten powder, vital wheat gluten powder, soy protein concentrate powder, soy isolate powder. It will be appreciated that the structure of the final product is better with the aforesaid at least one second protein powder. Moreover, the aforesaid at least one second protein powder further supplements the final product with protein, minerals such as iron, elastability, and so forth.

Optionally, the total weight of the powder mixture comprises:
from 20% up to 40% the first protein powder;
from 20% up to 40% at least one second protein powder;
from 20% up to 40% the water;
from 0.5% up to 1.5% the NaCl; and
from 2% up to 4% the at least one soluble fiber.

In this regard, optionally, each of the first protein powder and the second protein powder may for example be from 20, 25, 20 or 35% up to 25, 20, 25 or 40% of the powder mixture. Optionally, the first protein powder and the second protein powder together comprise 64.0-64.5% of the powder mixture. Optionally, the water may for example be from 20, 25, 20 or 35% up to 25, 20, 25 or 40%, preferably, 30%, of the powder mixture. Optionally, the NaCl may for example be from 0.5, 0.7, 0.9, 1.1 or 1.3% up to 0.7, 0.9, 1.1, 1.3 or 1.5%, preferably, 0.5, 0.6, 0.7, 0.8 or 0.9 up to 0.6, 0.7, 0.8, 0.9 or 1.0%, of the powder mixture. Optionally, the at least one soluble fiber may for example be from 4.0, 4.5, 5.0 or 5.5% up to 4.5, 5.0, 5.5 or 6.0%, preferably, 5%, of the powder mixture. Optionally, the at least one soluble fiber is pectin. More optionally, the pectin is obtained from apple, citrus fruits and vegetables, and so on. It will be appreciated that using pectin gives better structure to the final product.

Furthermore, the method comprises cutting the extruded mixture. The extruded mixture is harvested by cutting it into smaller blocks or pieces. In this regard, each cut may be achieved by a single movement of a cutter along a diection perpendicular to the die arranged at the end of the extrusion machine. It will be appreciated that a single cut cleanly removes a block of the extruded mixture for further use. Beneficially, cutting performed along the diection perpendicular to the die ensures preserving the quality and structure of the extruded mixture so produced. Subsequently, the block of the extruded mixture may be harvested and layered for production of the desired product, i.e. the HMMA.

Furthermore, the method comprises cooling the extruded mixture. It will be appreciated that the extruded mixture has inherently high temperature, such as for example room temperature or higher. Therefore, in order to increase the shelf life of the extruded mixture, the extruded mixture is cooled down in any suitable way known to a person skilled in the art.

Optionally, the cooled extruded mixture is stored the in containers, such as cans or pouch packets, which evacuated and sealed at its end. Beneficially, cooling and storing the extruded mixture prevents oxidation and infestation of the extruded mixture.

Optionally, the method further comprises freezing the extruded mixture. Optionally, the extruded mixture is frozen below −9.5° C. Beneficially, freezing the extruded mixture enhances the shelf life of the product even further. Moreover, freezing the extruded mixture prevents infestation of the extruded mixture by various pathogens.

### DETAILED DESCRIPTION OF THE DRAWINGS

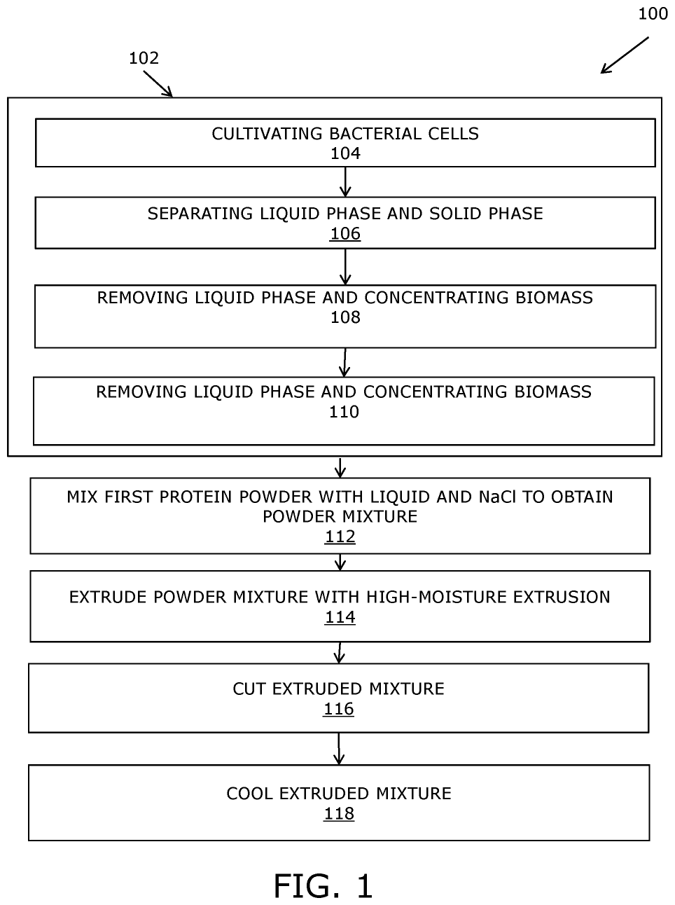

Referring to FIG. 1, there is shown a flowchart 100 illustrating steps of a method of producing a meat analogue food ingredient, in accordance with an embodiment of the present disclosure. A downstream process 102, comprises steps for producing a first protein powder. At step 104 bacterial cells are cultivated to obtain a biomass. At step 106 a liquid phase and a solid phase of the biomass are separated. At step 108 the biomass is concentrated by removing the liquid phase. At step 110, the biomass is dried to obtain the first protein powder.

At step 112, a first protein powder is mixed with a liquid and NaCl to obtain a powder mixture. At step 114, the powder mixture is extruded with high-moisture extrusion. At step 116, the extruded mixture is cut. At step 118, the extruded mixture is cooled.

The steps 102, 104, 106, 108, 110, 112, 114, 116 and 118 are only illustrative and other alternatives can also be provided where one or more steps are added, one or more steps are removed, or one or more steps are provided in a different sequence without departing from the scope of the claims herein.

It may be understood by a person skilled in the art that the FIG. 1 is merely an example for sake of clarity, which should not unduly limit the scope of the claims herein. The person skilled in the art will recognize many variations, alternatives, and modifications of embodiments of the present disclosure. In an example, the processes described in steps 102, 104, 106 and 108 may follow a different sequence to provide a final product, i.e. the meat analogue food ingredient.

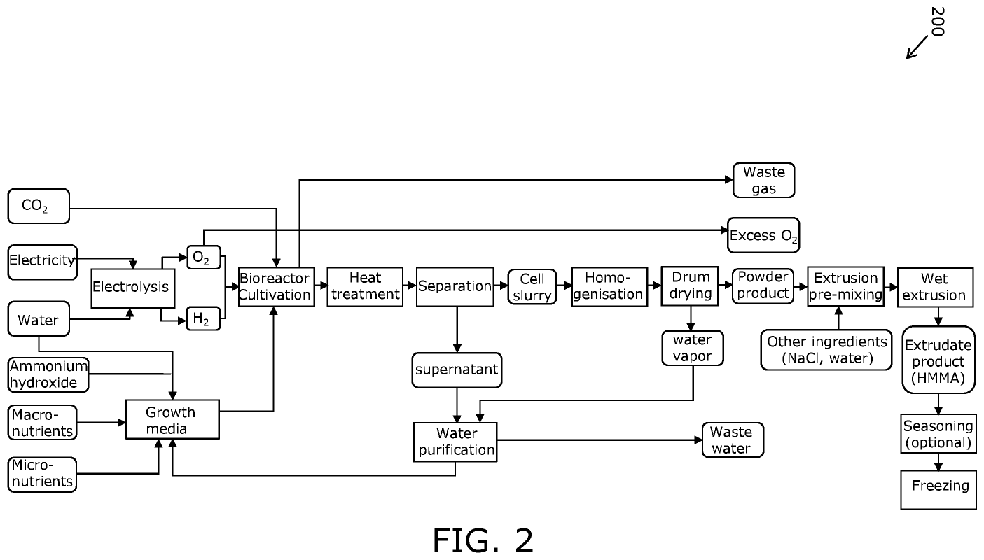

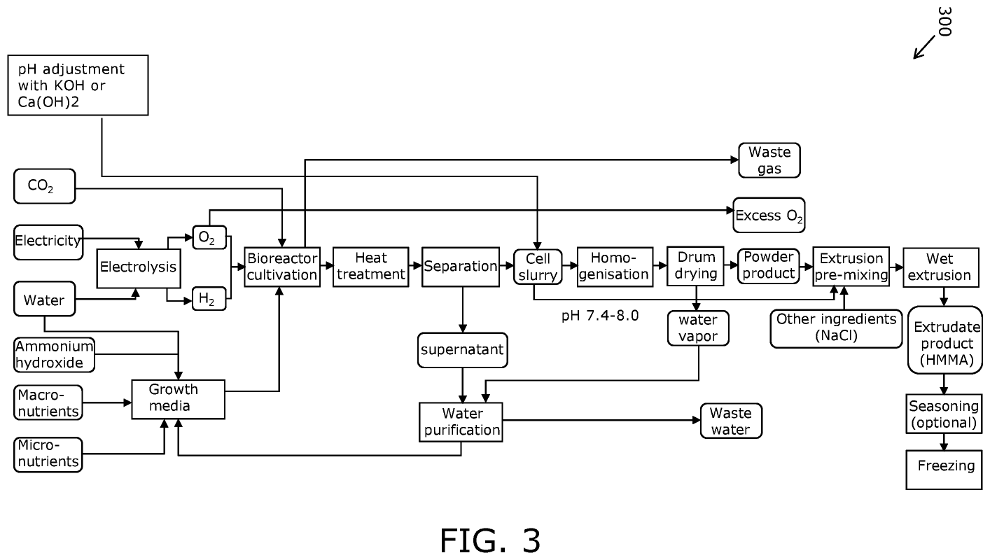

Referring to FIGS. 2, 3 and 4, there are shown flowcharts 200, 300 and 400, respectively, illustrating upstream and downstream processing of meat analogue food ingredient (referred to as high moisture meat analogue (HMMA) hereforth), in accordance with various embodiments of the present disclosure. As shown in FIGS. 2 and 3, the bacterial cells are subjected to bioreactor cultivation. The bioreactor cultivation requires supplying the bacterial cells with carbon dioxide gas, oxygen gas, hydrogen gas and growth media. Moreover, the oxygen gas and hydrogen gas are obtained by electrolysis of water using electricity. The water is further used to prepare a growth media that additionally comprises ammonium hydroxide, macronutrients and micronutrients, and is later sterilized. It will be appreciated that the bioreactor cultivation is performed at a predetermined conditions that facilate growth of the bacterial cells to have a high cell density of a biomass. It will be appreciated that during the bioreactor cultivation, water gas and excess oxygen gas is released in the atmosphere or recycled as required.

After achieving a desired high cell density of the biomass during bioreactor cultivation, the biomass is subjected to a heat treatment (namely, incubation) in a heat-exchanger, for example. Subsequently, the incubated biomass is concentrated by separation of a liquid phase from a solid phase of the biomass. The separated liquid phase or supernatant is subjected to purification and recycled to produce water and remove endotoxins therein. The solid phase of the biomass or the cell slurry is subjected to homogenization. The homogenized biomass is subjected to drum drying and the water vapour removed during drying process is directed for purification thereof. The powder product resulting from the drum drying is mixed with other ingredients, such as liquid, NaCl, at least one second protein powder, a soluble fiber, and so on, during extrusion pre-mixing. In this case, the powder mixture comprises the first protein powder and the second protein powder in an amount of 192 g (i.e. 64%), the amount of water is 90 g (i.e. 30%), the amount of NaCl is 3 g (i.e. 1%), the at least one soluble fiber is obtained from apple or citrus fruit in an amount of 15 g (i.e. 3%) to yield a total of 300 g of protein powder. The flour mix resulting from extrusion pre-mixing is subjected to high moisture extrusion (or wet extrusion) to yield the extrudated product high moisture meat analogue (or HMMA). The HMMA is cut into blocks or smaller pieces. The HMMA could optionally be mixed with seasoning and cooled or frozen to increase shelf-life thereof.

Moreover, as shown in FIG. 3, the cell slurry is mixed with a pH adjustor, such as potassium hydroxide (KOH) or calcium hydroxide (Ca(OH)₂) to obtain a pH of the cell sturry in a range of 7.4-8.0. It will be appreciated that for pH value lower than 7.0, the extruded product would fail to provide a meat-like texture upon extrusion. Moreover, the extrusion pre-mixing comprised mixing the powder product and NaCl only.

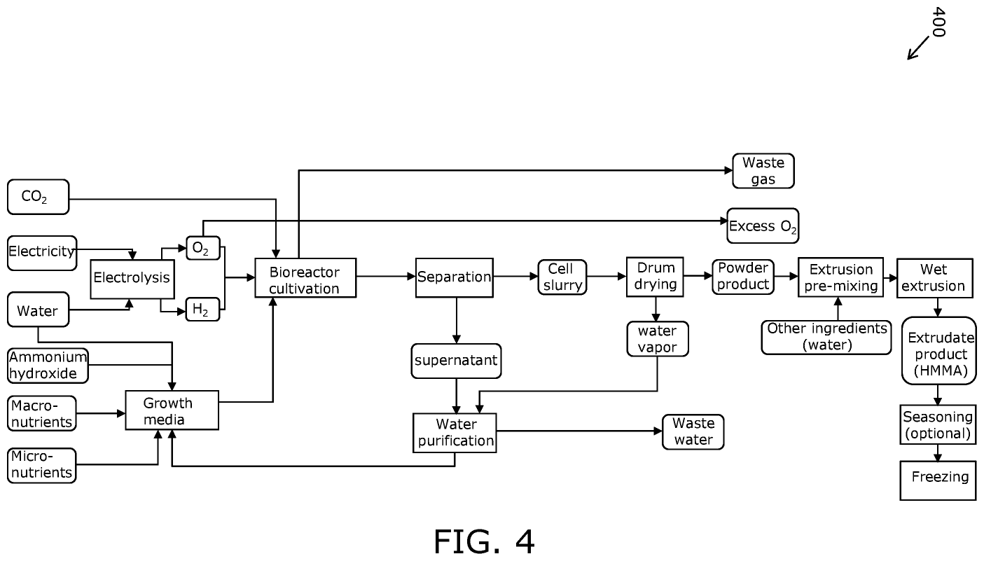

Furthermore, as shown in FIG. 4, the biomass is not subjected to heat treatment after harvesting thereof from the bioreactor cultivation, and the cell slurry is directly subjected to drum drying by skipping homogenization. In this case, the powder mixture comprises the first protein powder and the second protein powder in an amount of 120 g (i.e. 70%) and the amount of water is 51 g (i.e. 30%) to yield a total of 171 g of protein powder. Moreover, the extrusion pre-mixing comprised mixing the powder product and water only.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A method of producing a meat analogue food ingredient, the method comprising:
a downstream process comprising
cultivating bacterial cells to obtain a biomass,
separating a liquid phase and a solid phase of the biomass,
concentrating the biomass by removing the liquid phase, and
drying the biomass to obtain the first protein powder;
mixing a first protein powder with a liquid and NaCl to obtain a powder mixture;
extruding the powder mixture with high-moisture extrusion;
cutting the extruded mixture; and
cooling the extruded mixture.

2. A method according to claim 1 further comprising freezing the extruded mixture.

3. A method according to claim 1 further comprising adding at least one of selected from at least one second protein powder and at least one soluble fiber to the powder mixture before mixing.

4. A method according to claim 1, wherein the total weight of the powder mixture comprises:
from 20% up to 40% the first protein powder;
from 20% up to 40% at least one second protein powder;
from 20% up to 40% the water;
from 0.5% up to 1.5% the NaCl; and
from 2% up to 4% the at least one soluble fiber.

5. A method according to claim 2, wherein the at least one second protein powder is selected to be at least one of a pea isolate powder, wheat gluten powder, vital wheat gluten powder, soy protein concentrate powder, soy isolate powder.

6. A method according to claim 1, wherein the powder mixture is mixed in a mixer selected from at least one of a pre-conditioner, a flour mixer, a twin-screw extrusion machine.

7. A method according to claim 1, wherein high-moisture extrusion is carried out with following parameters:
torque from 1.0 Nm up to 1.3 Nm;
die pressure from 15 bars up to 18 bars;
die temperature from 140° C. up to 160° C.; and
melting temperature from 135° C. up to 155° C.

8. A method according to claim 1, wherein the liquid is selected to be at least one of a water, a protein slurry.

9. A method according to claim 8, wherein the protein slurry comprises from 90% up to 95% of water, from 1% up to 10% of a third protein powder.

10. A method according to claim 1, wherein the at least one of selected from the first protein powder and the third protein powder comprises an isolated bacterial strain deposited as VTT-E-193585 or a derivative thereof.

11. A method according to claim 1, wherein the downstream process further comprises incubating the biomass with a heat treatment at temperature from 55° C. up to 75° ° C. from 15 minutes up to 40 minutes.

12. A method according to claim 1, wherein the downstream process further comprises homogenizing the bacterial cells of the biomass before drying step.

13. A method according to claim 12, wherein the homogenizing is carried out for at least one run at pressure from 800 bars up to 2000 bars or preferably at pressure from 700 bars up to 1000 bars.

14. A method according to claim 12 further comprising filtering the homogenized bacterial cells of the biomass by at least one of selected from nanofiltration or ultrafiltration.

15. A method according to claim 12 further comprising adjusting the biomass pH to be from 7.4 up to 8.5 after at least one step of selected from separating the liquid phase or homogenizing.

16. A method according to claim 12, wherein the bacterial cells are cultivated by gas fermentation and the feed comprises at least one of selected from CO₂, CH₄, H₂, O₂, NH₃, at least one mineral.
